(19) United States
(12) Patent Application Publication
(10) Pub. No.: US 2014/0335546 A1
(43) Pub. Date: Nov. 13, 2014

(54) Devices and Methods for Determining the Risk of Developing a Serious Infection

(71) Applicant: G J CARROLL PTY LTD, Mount Lawley (AU)

(72) Inventor: Graeme Carroll, Mount Lawley (AU)

(21) Appl. No.: 13/891,429

(22) Filed: May 10, 2013

(51) Int. Cl. G01N 33/68

(52) U.S. Cl. CPC G01N 33/6893
USPC 435/7.92; 435/287.2

(57) ABSTRACT

A device for calculating risk of a patient developing a serious infection, the device comprising: a controller; storage storing electronic program instructions for controlling the controller; a display for displaying a user interface; and input means; wherein the controller is operable, under control of the electronic program instructions, to: receive input via the input means, the input comprising one or more values representing respective measures of one or more risk factors associated with biological sample(s) obtained from the patient; process the input to calculate the risk; and display an indication of the calculated risk via the display.

### TECHNICAL FIELD

A device and method for determining the risk of developing a serious infection in patients suffering from an autoimmune disease or inflammatory disorder. In particular the risk of developing a serious infection in patients that are suffering from rheumatoid arthritis.

### BACKGROUND ART

The following discussion of the background art is intended to facilitate an understanding of the present invention only. The discussion is not an acknowledgement or admission that any of the material referred to is or was part of the common general knowledge as at the priority date of the application.

The terms “autoimmune diseases” and “inflammatory disorders” cover a wide variety of diseases/disorders where the body's own immune system responds incorrectly to its own cells, tissues or organs, resulting in inflammation and damage. Common autoimmune diseases/inflammatory disorders include multiple sclerosis, autoimmune thyroid disease, type I diabetes, systemic lupus erythematosus, rheumatoid arthritis, systemic vasculitis, ulcerative colitis and Crohn's disease, psoriasis and psoriatic arthritis, ankylosing spondylitis and coeliac disease.

Rheumatoid Arthritis (RA) is a chronic form of disease, where the immune system attacks the body's own tissues, including the thin membrane that lines the joints (synovium). This results in a build-up of fluid in the joints, leading to pain and inflammation. There is no cure for RA and as such, long term treatment is required to maintain the health of the patient. Patients suffering from RA are at a higher risk of morbidity and mortality when compared to the unaffected population.

One issue that complicates the management of RA is the high risk in patients with RA of developing a serious infection(s), especially if the patient is also being treated with corticosteroids. That is, some RA patients are vulnerable to serious infections.

Although physicians are cognisant of the risk of serious infection in RA patients in general terms, the high frequency of serious infections in RA patients is not fully appreciated, partly because it is only over a long period of time that this becomes apparent (3-10 years) and more importantly because increasingly the prescribing physician is no longer the physician who takes direct responsibility for the actual treatment of the infections. Often the infection is treated by a general physician or an infectious diseases expert or an intensivist if the infection is so serious that intensive care unit expertise is required. In the case of serial infections, often it is different generalists or specialists who treat each serious infection episode and thus there is no continuity of care and hence diminished appreciation of the serial or recurrent nature of some infections. Hard decisions about whether to continue immunosuppressive or biologic therapies may not be taken by the treating Rheumatologist or primary care physician as a result of diminished appreciation of the impact of a serious infection. Thus there exists a need to develop a test to assess the vulnerability of a patient with RA to developing a serious infection.

Various risk calculators are available to establish the risk of developing cardiovascular disease or diabetes. These calculators take into account factors such as blood pressure, age, cholesterol and weight. The risk calculator puts all of these risk factors together to provide a risk score. Composite scores offer more powerful prediction.

In 2012, scientists at the Mayo clinic published a study describing a way of determining the risk of developing a serious infection in patients with RA. The risk factors included the number of previous serious infections, age, corticosteroid use, decrease white blood cell count, increases in erythrocyte sedimentation rate, signs of RA outside the joints, and other factors such as heart disease, diabetes, lung disease, heart failure, alcoholism, and vascular disease (Crowson et al., (2012). Arthritis & Rheumatism; 64(9): 2847-2855). These factors were assigned weightings to allow the calculation of a risk score. However, the resulting risk score is dependent on many factors that may be unreliable and based on the accuracy of collection of private information, old data and the honesty or recall of the patient. No biological markers were included in the preparation of the risk score.

Mannose binding lectin (MBL) is a serum protein, produced in the liver, which acts as a pattern recognition receptor. MBL recognises carbohydrate motifs in topographic arrays of glycoproteins on the surface of diverse microbes, including bacteria, viruses, fungi and parasites. MBL binding results in the killing of microorganisms by two mechanisms notably (i) activation of the complement attack complex with subsequent complement mediated microbial lysis, and (ii) phagocytosis of the microbe due to the opsonising effect of C3b production.

The MBL glycoprotein in human serum is the product of the MBL2 gene on chromosome 10. The MBL-2 gene is polymorphic. Gene mutations in the gene promoter region of the MBL2 gene and in the coding region exon 1 give rise to several common “null” alleles, combinations of which in conjunction with or without wild type alleles account for the diversity in MBL concentrations in serum. Approximately 5-8% of the Caucasian population have very low concentrations of MBL compatible with homozygous or compound heterozygous deficiency. Similar results are observed in other population groups. The deficiency in MBL has been associated with an increased susceptibility to infection, which is particularly evident in neonates with septicaemia, but also apparent in children with recurrent, usually serious infections and in adults with neutropaenia who require chemotherapy.

There exists a need to assess the susceptibility of individual patients suffering from an autoimmune disease or inflammatory disorder to develop a serious infection(s) using a simple test for a biological marker, and optionally together with one or more risk factors relevant to the health of the individual patient.

### SUMMARY OF INVENTION

Risk profiling and indeed the development of a clinical/biochemical risk calculator incorporating MBL concentrations may help to assist clinicians to estimate the relative risk of developing a serious infection in patients suffering from an autoimmune disease or inflammatory disease, such as Rheumatoid Arthritis (RA). It was surprisingly found that a deficiency in the MBL concentration in the serum of RA patients was a good indicator of the risk associated with the development of serious infections in RA patients.

In one aspect of the present invention there is provided a blood test to determine the risk of a patient suffering from rheumatoid arthritis developing a serious infection, wherein the blood test comprises measuring the concentration of MBL in the serum of a rheumatoid arthritis patient.

In a further aspect of the invention there is provided a method to determine the risk of developing a serious infection in a patient comprising measuring the concentration of MBL in the serum of the patient, and taking into consideration at least one other risk factor associated with the general health of the patient.

In another aspect of the present invention there is provided a method for assessing the risk of developing a serious infection in a patient suffering from an autoimmune disease or inflammatory disorder, comprising the steps of obtaining a biological fluid sample from the patient, measuring in the sample the concentration of mannose binding lectin, and correlating the concentration of mannose binding lectin to the risk of developing a serious infection.

In a further aspect of the present invention there is provided a method of predicting the risk of developing a serious infection in a patient suffering from an autoimmune disease or inflammatory disorder, wherein the method comprises the steps of: (a) determining for the patient the concentration of mannose binding lectin in a biological sample obtained from the patient; (b) determining for the patient at least one other risk factor; (c) predicting the risk of the patient developing a serious infection by correlating the risk factors determined in steps a) and b) with a predefined risk value associated with developing a serious infection.

In one embodiment of the present invention there is provided a method of predicting the risk of developing a serious infection in a patient suffering from an autoimmune disease or inflammatory disorder, wherein the method comprises the steps of: (a) determining for the patient the concentration of mannose binding lectin in a biological sample obtained from the patient; (b) determining for the patient at least one or more other risk factor selected from the group consisting of: the age of the patient, the gender of the patient, corticosteroid use, white blood cell count, the erythrocyte sedimentation rate, diabetes, lung disease, heart failure, alcohol usage, vascular disease, blood pressure, cholesterol, smoking status, kidney disease, presence of indwelling catheters, medication usage, neutropenia, and weight; and, (c) predicting the risk of the patient developing a serious infection by correlating the risk factors determined in steps a) and b) with a predefined risk value associated with developing a serious infection.

In another aspect of the invention, there is provided a device for calculating risk of a patient developing a serious infection, the device comprising: a controller; storage storing electronic program instructions for controlling the controller; a display for displaying a user interface; and input means; wherein the controller is operable, under control of the electronic program instructions, to: receive input via the input means, the input comprising one or more values representing respective measures of one or more risk factors associated with biological sample(s) obtained from the patient; process the input to calculate the risk; and display an indication of the calculated risk via the display.

The one or more values representing respective measures of one or more biological samples may comprise concentrations of one or more biological markers, and preferably a concentration of mannose binding lectin in serum of the patient. In such an embodiment, the processing may comprise correlating the concentration of mannose binding lectin to the risk of developing a serious infection.

The one or more values representing respective measures of one or more biological samples may comprise concentrations of one or more biological markers in addition to a concentration of mannose binding lectin. The one or more additional biological markers may be selected from the group consisting of: blood markers, bone or cartilage markers, synovial fluid markers, cerebrospinal fluid markers, inflammation markers, genetic markers, and radiological scores predicted from a biological marker.

The biological sample is preferably a fluid sample, such as, but not limited to blood, saliva, cerebrospinal fluid, synovial fluid or urine.

The input may additionally comprise one or more values representing respective measures of one or more other risk factors associated with the patient's health. In such an embodiment, the processing may comprise taking into consideration the one or more values representing respective measures of one or more risk factors associated with the patient's health. The consideration may be implemented by correlating each of the one or more values representing respective measures of one or more risk factors with a corresponding predefined risk value associated with developing a serious infection.

In embodiments of the invention, for each risk factor, there is a corresponding value range within which the value representing the measure of the risk factor must fall. The value range may be determined according to the impact of the risk factor on the likelihood of the patient developing a serious infection.

The one or more other risk factors may be selected from the group consisting of: age, gender, corticosteroid use, white blood cell count, erythrocyte sedimentation rate, diabetes, lung disease, heart failure, alcohol usage, vascular disease, blood pressure, cholesterol, smoking status, kidney disease, presence of indwelling catheters, medication usage, neutropenia, and weight.

The controller is preferably operable, under control of the electronic program instructions, to display a request for the input. The request may specify the input required.

The indication of the calculated risk may take the form of a predictive score. The processing may comprise calculating the predictive score by summing the corresponding risk values for each of the one or more risk factors. The indication may comprise a visual representation of the predictive score. In embodiments of the invention, the visual representation may comprise a plurality of colours associated with respective predictive scores or ranges of predictive scores. The colours may be proportional to the degree of risk associated with respective predictive scores.

The processing may comprise determining risk of developing a serious infection(s) by correlating the predictive score for an individual patient with the risk associated with that predicative score in accordance with a pre-determined probability distribution. In the pre-determined probability distribution, percentage weightings may be assigned to respective risk factors.

The patient may be a patient suffering from an autoimmune disease or inflammatory disorder. The autoimmune disease/inflammatory disorder may be selected from the group consisting of: multiple sclerosis, autoimmune thyroid disease, type I diabetes, systemic lupus, erythematosis, rheumatoid arthritis, systemic vasculitis, irritable bowel disease, ulcerative colitis, psoriasis, chronic obstructive pulmonary disease and Crohn's disease. Preferably the disease is rheumatoid arthritis.

The controller may comprise computer processing means.

The display, user interface and input means may be integrated, in a touchscreen for example. Alternatively, they may be discrete.

Preferably, the electronic program instructions comprise software. The device may comprise a smartphone having the software installed thereon. The software may be provided as a software application downloadable to the smartphone.

According to another aspect of the invention, there is provided a method for calculating risk of a patient developing a serious infection, the method comprising: storing electronic program instructions for controlling a controller; and controlling the controller via the electronic program instructions, to: receive input via an input means, the input comprising one or more values representing respective measures of one or more risk factors associated with biological sample(s) obtained from the patient; process the input to calculate the risk; and display an indication of the calculated risk via the display.

According to another aspect of the invention, there is provided a computer-readable storage medium on which is stored instructions that, when executed by a computing means, causes the computing means to perform the method for calculating risk of a patient developing a serious infection as hereinbefore described.

According to another aspect of the invention, there is provided a computing means programmed to carry out the method for calculating risk of a patient developing a serious infection as hereinbefore described.

According to another aspect of the invention, there is provided a data signal including at least one instruction being capable of being received and interpreted by a computing system, wherein the instruction implements the method for calculating risk of a patient developing a serious infection as hereinbefore described.

According to another aspect of the invention, there is provided a system for calculating risk of a patient developing a serious infection comprising the device for calculating risk of a patient developing a serious infection as hereinbefore described.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a graph setting out the number of serious infections per hundred patient years in MBL concentration subsets.

FIG. 2 is a schematic diagram of a device in accordance with an embodiment of the present invention;

FIG. 3 is a system diagram of a system comprising the device; and

FIG. 4 is a screen shot depicting a calculation function provided by the device.

### DISCLOSURE OF THE INVENTION

### General

Those skilled in the art will appreciate that the invention described herein is susceptible to variations and modification other than those specifically described. It is to be understood that the invention includes all such variations and modification. The invention also includes all of the steps, features, compositions and compounds referred to or indicated in the specification, individually or collectively and any and all combinations or any two or more of the steps or features.

The present invention is not to be limited in scope by the specific embodiment or examples described herein, which are intended for the purpose of exemplification only. Functionally equivalent products, compositions and methods are clearly within the scope of the invention as describe herein.

The entire disclosures of all publications (including patents, patent applications, journal article, laboratory manuals, book or other documents) cited herein are hereby incorporated by reference. No admission is made that any of the references constitute prior art or are part of the common general knowledge of those working in the field to which this invention relates.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

The term “serious infection” as used herein refers to an infection that leads to death, hospitalisation or requires intravenous antibiotics. Serious infections include, but not limited to bacterial infections, mycobacterium tuberculosis and other mycobacterial infections, invasive pneumococcal disease, septicaemia and bacteraemia, invasive bacterial infection after chemotherapy, neonatal septicaemia, meningitis, encephalitis, bone and joint sepsis, severe cutaneous infections including cellulitis, urosepsis, bowel and other GI tract infections, severe viral infections and opportunistic infections, especially fungal infections.

The term “risk factors” as used herein refers to any factor that may contribute to the health status of an individual patient. Risk factors include, but are not limited to mannose binding lectin, blood pressure, age, cholesterol, gender, smoking status, diabetes, kidney disease, heart disease, weight, use of corticosteroids, medication usage, white blood cell count, neutropenia, erythrocyte sedimentation rate, lung disease, alcohol usage, presence of indwelling catheters, and vascular disease.

The term “biological sample” as used herein refers to a sample obtained from the patient including, but not limited to, bone, cartilage, blood, derivatives of bloods, serum, synovial fluid, cerebrospinal fluid, interstitial fluid, urine, or saliva.

The term “biological fluid sample” as used herein refers to a fluid, including, but not limited to blood, derivatives of bloods, serum, synovial fluid, cerebrospinal fluid, interstitial fluid, urine, or saliva.

The term a “disease” or “disorder” as used herein refers to any autoimmune disease or inflammatory disorder including but not limited to multiple sclerosis, autoimmune thyroid disease, type I diabetes, systemic lupus erythematosus, rheumatoid arthritis, systemic vasculitis, ulcerative colitis and Crohn's disease, psoriasis and psoriatic arthritis, ankylosing spondylitis and coeliac disease.

A person skilled in the art would be aware that the terms “determining”, “assessing”, “calculating” or “predicting” when used in relation to evaluating the risk of developing a serious infection can be used interchangeably and have the same or similar meaning.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### DETAILED DESCRIPTION OF THE INVENTION

To date there is no reliable method available for a clinician, physician or medical practitioner to determine the likelihood that a patient suffering from an autoimmune disease or inflammatory disorder, such as rheumatoid arthritis will develop a serious infection(s).

The devices, systems and methods described herein are directed towards predicting the risk of developing a serious infection(s) in patients suffering from an autoimmune disease or inflammatory disorder. In particular, the method is directed towards predicting the risk of developing a serious infection in patients that are suffering from rheumatoid arthritis. In one embodiment, the method comprises measuring the concentration of mannose binding lectin in a biological sample obtained from an individual that may be at risk of developing a serious infection(s) and correlating the concentration of mannose binding lectin in the sample to a predictive score. Optionally, one or more other risk factors associated with the health of the individual may be included in the predictive score.

Mannose binding lectin (MBL) is a serum protein which recognises carbohydrate motifs in topographic arrays of glycoproteins on the surface of diverse microbes, including bacteria, viruses, fungi and parasites. The concentration of mannose binding lectin (MBL) in the serum of a healthy adult human subject is in the range of 1,300 to 3,000 or more ng/ml. An individual is considered to be MBL deficient if the concentration of MBL in the serum is determined to be less than 56 ng/ml. A deficiency in the concentration of MBL in the serum of patients has been suggested to be predictive of the risk of an individual suffering from an autoimmune disease or inflammatory disorder developing a serious infection(s).

The concentration of MBL can be measured in samples obtained from subjects by any methods known in the art. For example, the concentration of MBL can be measured by an automated ELIZA.

Thus in one embodiment of the present invention there is provided a blood test to determine the risk of a patient suffering from rheumatoid arthritis developing a serious infection, wherein the blood test comprises measuring the concentration of MBL in the serum of a rheumatoid arthritis patient.

A further embodiment of the present invention is a method to determine the risk of developing a serious infection in a patient comprising measuring the concentration of MBL in the serum of the patient, and taking into consideration at least one other risk factor associated with the general health of the patient.

In a preferred embodiment of the present invention, there is provided a method of predicting the risk of developing a serious infection in a patient suffering from an autoimmune disease or inflammatory disorder, wherein the method comprises the steps of: (a) determining for the patient the concentration of mannose binding lectin in a biological sample obtained from the patient; (b) determining for the patient at least one or more other risk factors; and, (c) predicting the risk of the patient developing a serious infection by correlating the risk factors determined in steps a) and b) with a predefined risk value associated with developing a serious infection.

In a preferred embodiment the risk factors are selected from selected from the group consisting of: the age of the patient, the gender of the patient, corticosteroid use, white blood cell count, the erythrocyte sedimentation rate, diabetes, lung disease, heart failure, alcohol usage, vascular disease, blood pressure, cholesterol, smoking status, kidney disease, presence of indwelling catheters, medication usage, neutropenia, and weight.

In one embodiment of the invention, the biological sample is blood, serum, saliva, cerebrospinal fluid, synovial fluid or urine.

In the present invention, the risk of developing a serious infection(s) may be determined by correlating the prediction score for an individual with the risk associated with that predication score in accordance with a pre-determined probability distribution.

In one embodiment of the present invention, the risk of developing a serious infection is significantly more frequent in patients where the serum MBL concentration is about 600 ng/mL or lower. When the concentration of MBL in serum is below 600 ng/ml and decreases in decrements of about 100 ng/ml, the probability of developing a serious infection becomes progressively stronger such that by a serum MBL concentration of 56 ng/mL is associated with a very high probability of developing a serious infection.

In a preferred embodiment, a value of between 0 and 20 may be allocated to reflect the risk attributable to the concentration of MBL in serum. For example, an MBL serum concentration of 56 ng/ml or less is allocated a value of 20. An MBL serum concentration of between about 100 to 600 ng/ml is allocated a value of 8, an MBL serum concentration of about 600 to about 1,300 ng/ml is allocated a value of 4, and an MBL serum concentration of greater than 1,300 ng/ml is allocated a value of 0. That is, a high value of 20 represents a high risk of developing a serious infection as a consequence of that risk factor, and contributes to the overall composite score, accordingly.

The measurement of variations in the concentration of MBL on its own or in combination with one or more risk factors associated with the health of an individual suffering from an autoimmune disease or inflammatory disorder, compared to a pre-determined predictive score provides a means to predict the risk of the individual developing a serious infection(s).

Determining a high risk of developing a serious infection(s) will assist the clinician or physician in preventative treatment, thereby assisting in alleviating the need for hospitalisation or intravenous antibiotics, or in extreme instances, avoiding death of the individual.

Serious infections are very important because in many cases they are life threatening and sadly in a significant proportion of cases result in the death of the patient. If a serious infection is foreseeable, it is most often preventable and usually eminently treatable with good outcomes for the patient, especially if recognised promptly and treated appropriately.

The predictive score generated through the systems and methods of the present invention provide patients with the necessary information to be aware of the risks that accompany the treatments prescribed to them by their clinician. Serious infections need to be catalogued and monitored with a view to reviewing treatment where appropriate and strategies need to be developed to minimise risk and maximise outcomes.

The devices, systems and methods of the present invention provide the patient with the ability to be pro-active and to monitor their own welfare in regard to these issues. The risk calculator defined in the present invention is an empowering tool for both the clinician and the patient. Likewise, a risk calculator can be of considerable assistance to primary care physicians who need the assistance it can provide to estimate patient risk. Many general practitioners are familiar with cardiovascular risk factors and the use of CV risk calculators. Furthermore the availability of a calculator on various health websites, such as the National Rheumatology Professional websites, primary care practice desktop computers and general practitioner websites as well as on mobile devices as downloadable applications and in other formats would make the determination of serious infection risk accessible and readily calculable within an acceptable time-frame at the point of care. This would assist in the development of individual action plans for patients at high risk of developing a serious infection.

### EXAMPLES

### Example 1

### Measurement of MBL in Patients Suffering with Rheumatoid Arthritis

To determine whether MBL deficiency is an independent risk factor for the development of a serious infection in patients suffering with Rheumatoid Arthritis (RA), we investigated 229 patients with RA who satisfied the 1987 ACR criteria for the diagnosis of RA [median duration, 10 years (range 1-58 years)]. The median age was 62 years (22-88), 69% were female and 70% were rheumatoid factor positive. (15%) were current smokers, 9% had COLD, 7% had either T1 or T2 DM and 12% were receiving corticosteroids at a maintenance dose of 5 mg per day or more. None of the patients included in this study were neutropaenic.

From 1 Jan. 2007 to 31 Mar. 2013, details concerning serious infections which by definition required either hospitalization or intravenous antibiotic therapy or both were obtained by regular questioning about serious infections at the time of clinical review every 3 to 6 months throughout the duration of the study and by a systematic audit of hospital admission records in all of the 229 participants.

MBL was measured by an automated ELISA in a single laboratory. In 14 patients the MBL was measured at more than 1 time point. The Intra Class Coefficient (ICC) was calculated to assess the reliability of the MBL assay. The ICC value using a two way random effect model was found to be 0.935, 95% CI 0.674-0.982. This indicates a high degree of reliability. Although no local population data for MBL concentrations was available, comparisons with published data from comparable populations indicated a similar distribution of results. About 25% of participants had MBL concentrations below 400 ng/mL.

Single serious infections were observed in 38 (16.6%) of the 229 participants and multiple serious infections (two or more, median=2, range 2-6) were observed in 15 patients (6.5%). Amongst the synthetic DMARD subset (n=98), 20 participants (20.4%) developed a single serious infection and 6 (6.1%) developed multiple serious infections, whereas in the biologic recipients (n=131), 18 patients (13.7%) developed a single serious infection and 9 patients (6.9%) developed multiple serious infections.

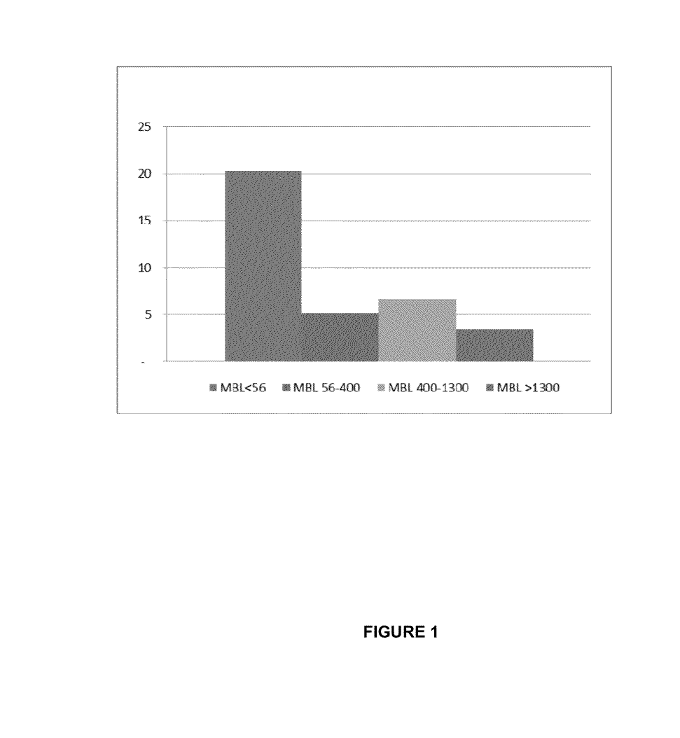

In the biologic recipient group, the rate of serious infections per hundred patient years is shown for MBL concentration subsets in FIG. 1. Whilst the rate for the entire group of biologic therapy recipients was 6.8 serious infections per 100 patient years, that for participants with very low MBL concentrations (<56 ng/mL, n=19, 14.5%) was 20 per hundred patient years (P=0.0001).

Amongst the full cohort (n=229) and as in other studies of synthetic DMARD and biologic therapies, pneumonia and LRTIs were by far the most common serious infection accounting for just under half the total number of serious infections (45.3%). Skin infections of all types accounted for 20.8%, bone and joint sepsis for 11.3%, bowel infections for 9.4% and all others for 13.2%. Serious infections occurred in 23 of the 28 participants who were receiving corticosteroids (CS), usually Prednisolone at doses of 5 mg per day or greater (82.1%, OR=23.7). Multivariate analysis showed that even when account is taken of CS usage, MBL deficiency is still a major risk factor for single and multiple serious infections (OR=5.33 for at least one and 40.8 for multiple serious infections). In the biologic recipient group, participants with an MBL<56 ng/mL had a 4.65 times greater risk for serious infection (IRR=4.65 CI 2.40-9.02, P<0.0001). The IRR was determined by a Poisson regression model.

The precise frequency of serious infections in RA is unknown. Furthermore, the ethical imperative to treat RA makes it unlikely now that contemporary rates of serious infections in native disease will be determined. Serious infections were observed far more commonly in RA irrespective of the use of biologic therapy. For example the frequency of serious infections over the 6 year period of observation in the RA cohort was ten times higher at 23% compared to 2.2% in a parallel cohort of patients with psoriatic arthritis (PsA), where only 1 of 44 patients developed an serious infection (P=0.0007) over a comparable time period (same study methodology, full data not shown).

We believe there is a significant increase in the background rate of serious infection in RA. Hitherto, there has been no biochemical test available to help inform clinicians about the risks over and above those apparent clinically, such as advanced age and corticosteroid usage. Our findings indicate that very low concentrations of MBL are an important risk factor for both single and multiple or recurrent serious infections in this disease. Furthermore MBL deficiency has a higher OR for serious infections than does age, maintenance CS usage, tobacco consumption, COLD or diabetes mellitus.

### Example 2

### Risk Calculator

In the drawings, like features have been referenced with like reference numbers.

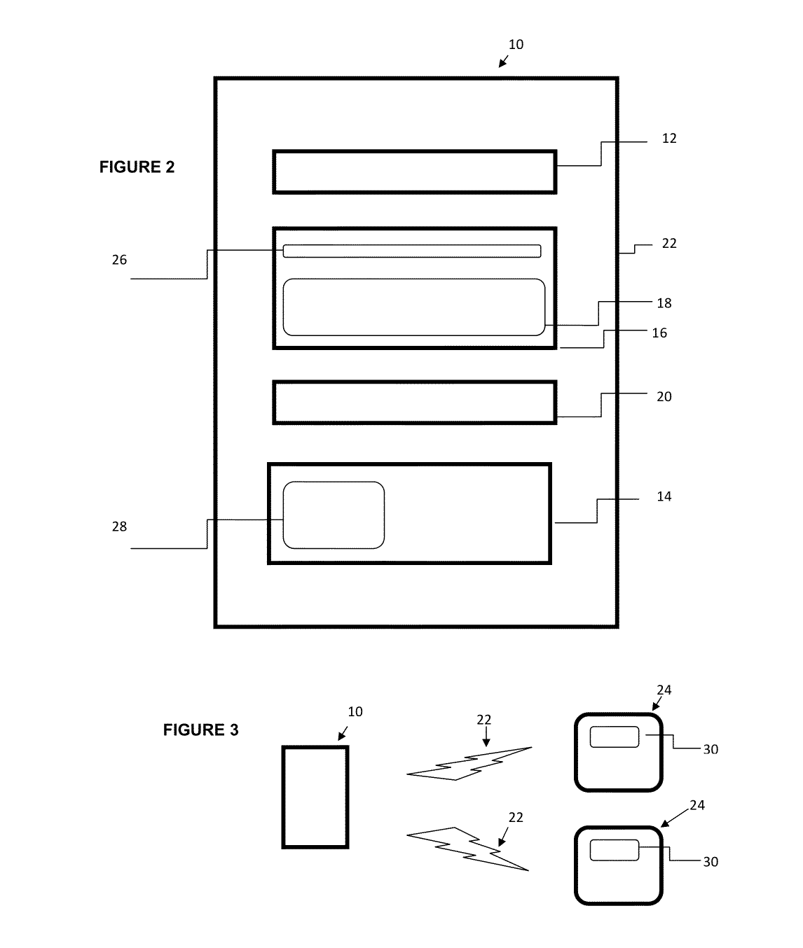

In FIG. 2, there is depicted an embodiment of a device 10 for calculating risk of a patient developing a serious infection in accordance with an aspect of the present invention.

The device 10 comprises a plurality of components, subsystems or modules operably coupled via appropriate circuitry and connections to enable the device 10 to perform the functions and operations herein described. The device 10 comprises suitable components necessary to receive, store and execute appropriate computer instructions such as a method for calculating risk of a patent developing a serious infection in accordance with an aspect of the present invention.

Particularly, the device 10 comprises computing means which in this embodiment comprises a controller 12 and storage comprising a storage means, medium or device 14 for storing electronic program instructions for controlling the controller 12, and information and/or data; a display 16 for displaying a user interface 18; and a user input means 20; all housed within a container or housing 22.

As will be described in further detail, the controller 12 is operable, under control of the electronic program instructions, to: receive input via the user input means 20, the input comprising one or more values representing respective measures of one or more biological samples obtained from the patient; process the input to calculate the risk; and display an indication of the calculated risk via the display 16.

The controller 12 comprises processing means in the form of a processor.

The storage device 14 comprises read only memory (ROM) and random access memory (RAM).

The device 10 is operable to communicate via one or more communications link(s) 22, which may variously connect to one or more remote devices 24 such as servers, personal computers, terminals, wireless or handheld computing devices, landline communication devices, or mobile communication devices such as a mobile (cell) telephone. At least one of a plurality of communications links 22 may be connected to an external computing network through a telecommunications network.

The device 10 is capable of receiving instructions that may be held in the ROM or RAM and may be executed by the processor. The processor is operable to perform actions under control of electronic program instructions, as will be described in further detail below, including processing/executing instructions and managing the flow of data and information through the device 10.

The electronic program instructions are provided via a single software application (“app”) or module which may be referred to as a risk calculator app. In the embodiment described, the app can be downloaded from a website (or other suitable electronic device platform) or otherwise saved to or stored on the storage device 14 of the device 10.

In preferred embodiments of the invention, the device 10 comprises a smartphone such as that marketed under the trade mark IPHONE® by Apple Inc, or by other provider such as Nokia Corporation, or Samsung Group, having Android, WEBOS, Windows, or other Phone app platform. Alternatively, the device 10 may comprise other computing means such as a personal, notebook or tablet computer such as that marketed under the trade mark IPAD® or IPOD TOUCH® by Apple Inc., or by other provider such as Hewlett-Packard Company, or Dell, Inc, for example.

The software app, or software, electronic instructions or programs for the computing components of the device 10, can be written in any suitable language, as are well known to persons skilled in the art. For example, for operation on a device 10 comprising an PHONED smartphone, the software app may be written in the Objective-C language. In embodiments of the invention, rather than being a single software app, the electronic program instructions may comprise a set or plurality of software, electronic instructions or programs and can be provided as stand-alone application(s), via a network, or added as middleware, depending on the requirements of the implementation or embodiment.

The device 10 also includes an operating system which is capable of issuing commands and is arranged to interact with the software app to cause the device to carry out the respective steps, functions and/or procedures in accordance with the embodiment of the invention described herein. The operating system may be appropriate for the device 10. For example, in the case where the device 10 comprises an PHONED smartphone, the operating system may be iOS.

In alternative embodiments of the invention, the software may comprise one or more modules, and may be implemented in hardware. In such a case, for example, the modules may be implemented with any one or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA) and the like.

The computing means can be a system of any suitable type, including: a programmable logic controller (PLC); digital signal processor (DSP); microcontroller; personal, notebook or tablet computer, or dedicated servers or networked servers.

The processor can be any custom made or commercially available processor, a central processing unit (CPU), a data signal processor (DSP) or an auxiliary processor among several processors associated with the computing means. In embodiments of the invention, the processing means may be a semiconductor based microprocessor (in the form of a microchip) or a macroprocessor, for example.

In embodiments of the invention, the storage means, medium or device can include any one or combination of volatile memory elements (e.g., random access memory (RAM) such as dynamic random access memory (DRAM), static random access memory (SRAM)) and non-volatile memory elements (e.g., read only memory (ROM), erasable programmable read only memory (EPROM), electronically erasable programmable read only memory (EEPROM), programmable read only memory (PROM), tape, compact disc read only memory (CD-ROM), etc.). The storage means, medium or device may incorporate electronic, magnetic, optical and/or other types of storage media. Furthermore, the storage medium can have a distributed architecture, where various components are situated remote from one another, but can be accessed by the processing means. For example, the ROM may store various instructions, programs, software, or applications to be executed by the processing means to control the operation of the device 10 and the RAM may temporarily store variables or results of the operations.

The use and operation of computers using software applications is well-known to persons skilled in the art and need not be described in any further detail herein except as is relevant to the present invention.

Furthermore, any suitable communication protocol can be used to facilitate communication between any subsystems or components of the device 10, and the device 10 and other devices or systems, including wired and wireless, as are well known to persons skilled in the art and need not be described in any further detail herein except as is relevant to the present invention.

Where the words “store”, “hold” and “save” or similar words are used in the context of the present invention, they are to be understood as including reference to the retaining or holding of data or information both permanently and/or temporarily in the storage means, device or medium for later retrieval, and momentarily or instantaneously, for example as part of a processing operation being performed.

Additionally, where the terms “system”, “device”, and “machine” are used in the context of the present invention, they are to be understood as including reference to any group of functionally related or interacting, interrelated, interdependent or associated components or elements that may be located in proximity to, separate from, integrated with, or discrete from, each other.

Furthermore, in embodiments of the invention, the word “determining” is understood to include receiving or accessing the relevant data or information.

In the embodiment of the invention, the display 16 for displaying the user interface 18 and the user input means 20 are integrated in a touchscreen 26. In alternative embodiments these components may be provided as discrete elements or items.

The touchscreen 24 is operable to sense or detect the presence and location of a touch and/or gesture within a display area of the device 10. Sensed “touchings” of the touchscreen 24 and/or “gestures” are inputted to the device 10 as commands or instructions and communicated to the controller 12. It should be appreciated that the user input means is not limited to comprising a touchscreen, and in alternative embodiments of the invention any appropriate device, system or machine for receiving input, commands or instructions may be used, including, for example, a keypad or keyboard, a pointing device, or composite device.

In the embodiment, the controller 12 is operable, under control of the electronic program instructions, to display a request for the input, specifying the input required, via the touchscreen 24.

In the embodiment, the one or more values representing respective measures of one or more biological fluid samples inputted to the device 10 comprise a value representing a concentration of mannose binding lectin measured in serum of the patient. The concentration of mannose binding lectin measured in serum of the patient may be considered a first risk factor associated with the patient's health.

In alternative embodiments, the one or more values representing respective measures of one or more biological fluid samples may comprise values representing concentrations of one or more biological markers in addition to a concentration of mannose binding lectin. The one or more additional biological markers may be selected from the group consisting of: bone or cartilage markers, synovial fluid markers, other inflammation markers, genetic markers, and radiological scores. These may be considered as additional risk factors associated with the patient's health.

In the described embodiment, the input additionally comprises one or more values representing respective measures of one or more other risk factors associated with the patient's health.

The processing comprises correlating the inputted value for the concentration of mannose binding lectin and each of the inputted values representing respective measures of the one or more risk factors with a respective corresponding predefined risk value associated with developing a serious infection.

In the embodiment, for each risk factor, there is a corresponding value range within which the value representing the measure of the risk factor must fall. The value range may be determined according to the impact of the particular risk factor on the likelihood of the patient developing a serious infection.

The indication of the calculated risk displayed has the form of a predictive score in the embodiment. The processing comprises calculating the predictive score by summing the corresponding risk values for the concentration of mannose binding lectin and each of the risk factors.

In alternative embodiments of the invention, the processing may comprise additional and/or alternative statistical methods to those described herein.

In the described embodiment, the risk factors, and their associated weightings, include: (1) patient age (high weightings for neonates and young infants, moderate weighting for children, low weighting for young adults 20-40, incrementally higher weightings for middle aged, and still higher weightings for the old and very old); (2) previous serious infections of the patient; (3) corticosteroid use by the patient (especially long term maintenance CSs above 5 mg daily); (4) neutropaenia (especially if chronic and related to diseases, such as malignancy, autoimmune disorders or post-chemotherapy); (5) mannose binding lectin deficiency (especially if MBL is <400 ng/mL); and (6) co-morbidities (bronchiectasis (6), chronic obstructive lung disease (2), current smoker (2), high alcohol consumer (2), T1 or T2 diabetes (2), cancer necessitating radiotherapy or chemotherapy in the past 10 years (2)).

These six categories have been determined on the basis of statistically significantly increased odds ratios for serious infection in the case of age, CS usage and MBL deficiency in a study by the inventor. Although low neutrophil counts are known to predispose to serious infection, this has not been widely accepted in RA.

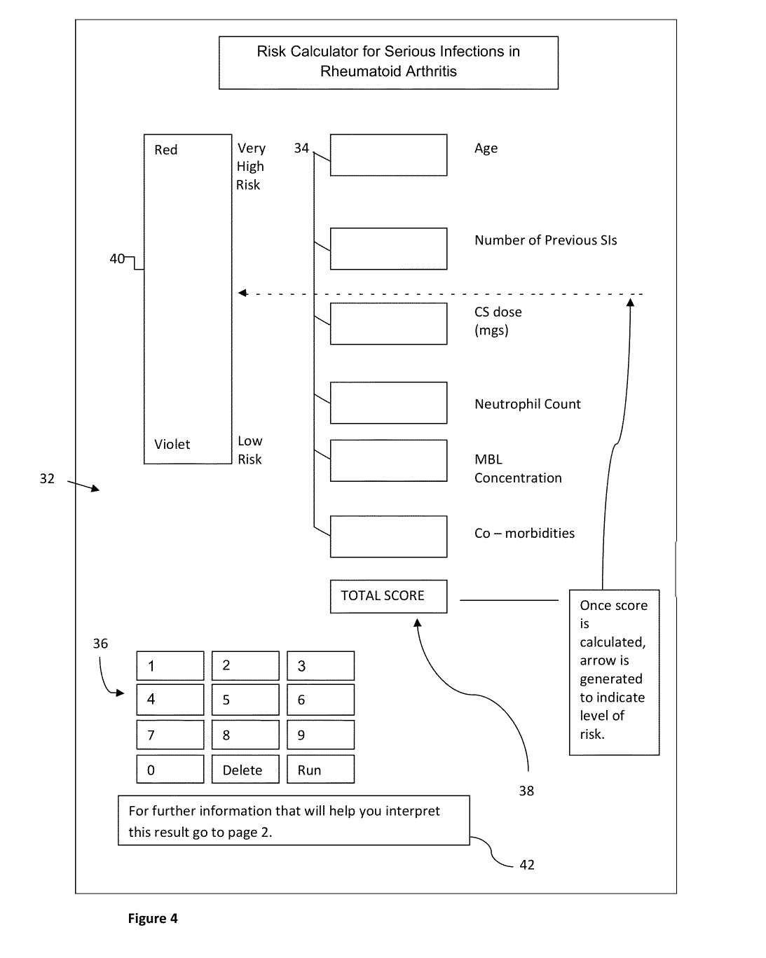

As will be described in further detail, the total score is interpreted qualitatively as depicted on the coloured bar portion 40 of the screen 32 depicted in FIG. 4 and a quantitative interpretation is provided which may be, for example a total score of less than 20, being associated with a low risk, 20-40 associated with a moderate risk, and 40-60 associated with a high risk (approximately a 30% likelihood of a serious infection within 5 years), a total score of 60-80 associated with a very high risk (at least a 50% likelihood of developing a serious infection within 5 years), and a total score of 80 plus associated with an extreme risk (at least a 75% likelihood of a developing a serious infection within 5 years).

In one example, any patient with a risk score greater than 40 should be encouraged to request, or be offered, a clinical review dedicated to the assessment of risk factors for infection. Amongst other issues, their treatment may be modified to reduce the risk of developing a serious infection. In addition, the vaccination status of the patient should be reviewed and a customised “action plan” should be developed so that the patient knows what action to take in the event of a suspected infection. Possibly, a patient at high risk of developing a serious infection should be provided with a Medic Alert bracelet or equivalent as a reminder to the patient of their high risk of developing a serious infection.

In embodiments of the invention, a weighting factor may be applied. This may take the form of subjecting the value inputted for a particular risk factors as a multiplication factor. For example the corticosteroid (CS) dose in milligrams (mg) is multiplied by a factor of two (2). Thus a CS dose of 12.5 mg daily for example will give rise to a score of 25. The CS score is capped at 30 so that a dose of 25 mg daily will only give rise to a score of 30. Thirty percent (30%) of the final total score may be achieved on the basis of CS usage alone. This is the main component of the calculated score reflecting the high odds ratio of 23.7 for CS usage. In contrast, the highest score possible for the age component is only 8 as is the case for example in an 83 year old, reflecting an odds ratio of 1.04. Other than these weightings there is no other component to the total score. In the embodiment.

In the embodiment, each of the six factors (including the concentration of mannose binding lectin) are scored approximately as follows:
1. X out of 8
2. X out of 16
3. X out of 30
4. X out of 10
5. X out of 20
6. X out of 16

For example, a patient at high risk may score as follows:
1. 5 for age 50
2. 12 for 2 previous serious infections
3. 20 for use of 10 mg of CS per day for more than 3 months
4. 0 for no neutropaenia
5. 20 for an MBL<56 ng/mL (compatible with severe deficiency)
6. 2 for T2DM
   Total score=59

Whereas a patient at low risk of infection may score as follows:
1. 4 for age 40
2. 0 for previous SIs
3. 0 for no use of CS
4. 0 for no neutropaenia
5. 0 for an MBL>1300 ng/mL
6. 2 for current smoker
   Total score=6

In the embodiment, the predictive score is proportional to the risk of infection. That is, the higher the total score, the higher the risk of infection.

In alternative embodiments, additional and/or alternative risk factors may be considered in the processing, including risk factors selected from the group consisting of: age, gender, corticosteroid use, white blood cell count, erythrocyte sedimentation rate, diabetes, lung disease, heart failure, alcohol usage, vascular disease, blood pressure, cholesterol, smoking status, kidney disease, presence of indwelling catheters, medication usage, neutropenia, and weight.

Information and/or data regarding or associated with the biological fluid sample(s) and other risk factors, such as their respective relevance and degree to which they are to be taken into account (i.e. weighting) in determining the risk (i.e. their impact on the likelihood of the patient developing a serious infection) and prescribed value ranges, are stored in the storage device 14. In alternative embodiments of the invention, additional or alternative information or data may be stored.

At least some of the information and/or data are stored or saved in a database 28 or databank residing on the storage device 14 and accessible by the controller 12 under control of the risk calculator app. These may be installed as part of the risk calculator application. The controller 12 is arranged to interact with the database 28 to cause the device 10 to carry out the respective steps, functions and/or procedures in accordance with the embodiment of the invention described herein.

Details of others of the one or more risk factors are stored or saved remotely, for example in one or more remote database modules 30 residing on respective storage of one or more remote systems or devices 24 and accessible by the device 10 via the one or more communications link(s) 22. The controller 12 is arranged to facilitate user interaction with the one or more remote databases 30, to make the remotely stored content available for free or on payment of a fee according to a fee schedule.

It will be understood that the database(s) may reside on any suitable storage device, which may encompass solid state drives, hard disc drives, optical drives or magnetic tape drives. The database(s) may reside on a single physical storage device (as in the embodiment described) or may be spread across multiple storage devices or modules.

The database 28 is coupled to the controller 12 and in data communication therewith in order to enable information and data to be read to and from the database 28 as is well known to persons skilled in the art. Any suitable database structure can be used, and there may be one or more than one database. In embodiments of the invention, the database 28 can be provided locally as a component of the device 10 (such as in the storage device 14) or remotely such as on a remote server, as can the electronic program instructions, and any other data or information to be gathered and/or presented. In an embodiment, several computers or devices can be set up in this way to have a network client-server application.

Once the risk calculator app is installed on the device 10, the controller 12 is operable, under control of the risk calculator app, to present, via the touchscreen 24, a sequence of electronic pages, screens and forms to a user or operator of the device 10 allowing for the inputting or capture of information, data, instructions and commands pertinent to a risk to be determined.

The above and other features and advantages of the embodiment of the invention will now be further described with reference to the device 10 in use.

A user, who is typically a medical practitioner/clinician, installs and executes the risk calculator app of the device 10.

The user then interfaces with the device 10 and provides user instructions via the touchscreen 24.

FIG. 4 of the drawings shows an example of what may be referred to a “Calculation” screen 32 displayed via the touchscreen 24. It will be appreciated that this is an example screen shot of the embodiment. In embodiments of the invention, this may be altered to suit user demand or feedback or to improve functionality, for example, and so other screens have a different visual appearance are possible.

Interface elements in the form of a plurality of data entry boxes 34, each corresponding to a respective one of the risk factors as hereinbefore described, are provided on the Calculation screen allowing the user to input values required for the calculation via use of a keypad 36. In addition to keys representing numerals 0 to 9, the keypad additional comprises a “Delete” key facilitating the deletion of incorrectly inputted values, and a “Run” key, activation of which initiates the calculation processing.

Once the values have been entered, and the Run key activated, the device 10 is operable to process the values as hereinbefore described to calculate the risk of the patient developing a serious infection(s).

The calculated predictive score for the patient arising from the processing is displayed in a “Total Score” portion 38 of the screen 32. Additionally, as herein before described, a visual representation of the predictive score is provided in a colour bar portion 40 of the screen 32, in which the device is operable to display a colour proportional to the degree of risk associated with the calculated predictive score to provide a quantitative interpretation of it. Presenting the results in this way will assist the clinician or physician in communicating the result to the patient, to promote better understanding of the risk of developing a serious infection.

An additional screen, providing further information regarding how to interpret the result, may be navigated to via an interface element in the form of an “Information Screen” button 42.

Calculating and providing the predictive score in this manner assists the user in expeditiously treating the patient.

### Example 3

### Treatment of Serious Infections in RA Patients

Based on the above findings we propose the use of MBL, and/or recombinant human MBL (rhMBL) to prevent multiple serious infections in RA patients vulnerable to recurrent serious infections. As noted in example 1 above, 86% RA patients had MBL concentrations less than 1000 ng/mL. In addition it may be appropriate to consider the prophylactic use of MBL or rhMBL in RA patients refractory to single biologic DMARDS who may be capable of responding to combination biologic therapy, since the major impediment to this therapeutic approach to date has been the high risk for serious infection.

1. A method for assessing the risk of developing a serious infection in a patient suffering from an autoimmune disease or inflammatory disorder, comprising the steps of obtaining a biological sample from the patient, measuring in the sample the concentration of mannose binding lectin, and correlating the concentration of mannose binding lectin to the risk of developing a serious infection.

2. A method to determine the risk of developing a serious infection in a patient comprising measuring the concentration of MBL in the serum of the patient, and taking into consideration at least one other risk factor associated with the health of the patient.

3. A method of predicting the risk of developing a serious infection in a patient suffering from an autoimmune disease or inflammatory disorder, wherein the method comprises the steps of: (a) determining for the patient the concentration of mannose binding lectin in a biological sample obtained from the patient; (b) determining for the patient at least one other risk factor; and (c) predicting the risk of the patient developing a serious infection by correlating the risk factors determined in steps a) and b) with a predefined risk value associated with developing a serious infection.

4. A device for calculating risk of a patient developing a serious infection, the device comprising: a controller; storage storing electronic program instructions for controlling the controller; a display for displaying a user interface; and input means; wherein the controller is operable, under control of the electronic program instructions, to: receive input via the input means, the input comprising one or more values representing respective measures of one or more risk factors associated with biological sample(s) obtained from the patient; process the input to calculate the risk; and display an indication of the calculated risk via the display.

5. A method for calculating risk of a patient developing a serious infection, the method comprising: storing electronic program instructions for controlling a controller; and controlling the controller via the electronic program instructions, to: receive input via an input means, the input comprising one or more values representing respective measures of one or more risk factors associated with biological sample(s) obtained from the patient; process the input to calculate the risk; and display an indication of the calculated risk via the display.

6. The method or the device according to any one of claims 1 to 5, wherein the disease or disorder is selected from the group consisting of: multiple sclerosis, autoimmune thyroid disease, type I diabetes, systemic lupus erythematosus, rheumatoid arthritis, systemic vasculitis, ulcerative colitis and Crohn's disease, psoriasis and psoriatic arthritis, ankylosing spondylitis and coeliac disease.

7. The method or the device according to any one of claims 1 to 6, wherein the risk factor is selected from the group consisting of: the age of the patient, the gender of the patient, corticosteroid use, white blood cell count, the erythrocyte sedimentation rate, diabetes, lung disease, heart failure, alcohol usage, vascular disease, blood pressure, cholesterol, smoking status, kidney disease, presence of indwelling catheters, medication usage, neutropenia, and weight.

8. The method or the device according to any one of claims 1 to 7, wherein the biological sample is obtained from the group consisting of: bone, cartilage, synovial fluid, cerebrospinal fluid, blood, saliva or urine.

9. The device according to claim 4 or claim 5, wherein the one or more values representing respective measures of one or more biological samples may comprise concentrations of one or more biological markers.

10. The device according to claim 9, wherein the one or more values is a concentration of mannose binding lectin in serum of the patient.

11. The device according to claim 9 or claim 10, wherein the processing may comprise correlating the concentration of mannose binding lectin to the risk of developing a serious infection.

12. The device according to any one of claims 4-11, wherein one or more additional biological markers are selected from the group consisting of: blood makers, bone or cartilage markers, synovial fluid markers, cerebrospinal fluid markers, inflammation markers, genetic markers, and radiological scores predicted from a biological marker.

13. The device according to any one of claims 4-12, wherein the input may additionally comprise one or more values representing respective measures of one or more other risk factors associated with the patient's health.

14. The device according to claim 13, wherein the processing may comprise taking into consideration the one or more values representing respective measures of one or more risk factors associated with the patient's health.

15. The device according to claim 14, wherein the consideration may be implemented by correlating each of the one or more values representing respective measures of one or more risk factors with a corresponding predefined risk value associated with developing a serious infection.

16. The device according to any one of claims 4-15, wherein for each risk factor, there is a corresponding value range within which the value representing the measure of the risk factor must fall.

17. The device according to claim 16, wherein the value range is determined according to the impact of the risk factor on the likelihood of the patient developing a serious infection.

18. The device according to any one of claims 4-17, wherein the controller is operable, under control of the electronic program instructions, to display a request for the input.

19. The device according to claim 18, wherein the request may specify the input required.

20. The device according to any one of claims 4-19, wherein the indication of the calculated risk may take the form of a predictive score.

21. The device according to any one of claims 4-20, wherein the processing comprises calculating the predictive score by summing the corresponding risk values for each of the one or more risk factors.

22. The device according to claim 20 or claim 21, wherein the indication comprises a visual representation of the predictive score.

23. The device according to any one of claims 4-22, wherein the visual representation comprises a plurality of colours associated with respective predictive scores or ranges of predictive scores.

24. The device according to claim 23, wherein the colours are proportional to the degree of risk associated with respective predictive scores.

25. The device according to any one of claims 4-24, wherein the processing comprises determining risk of developing a serious infection(s) by correlating the predictive score for an individual patient with the risk associated with that predicative score in accordance with a pre-determined probability distribution.

26. The device according to claim 25, wherein the pre-determined probability distribution, percentage weightings may be assigned to respective risk factors.

27. The device according to any one of claims 4-26, wherein the controller comprises computer processing means.

28. The device according to any one of claims 4-27, wherein the display, user interface and input means are integrated.

29. The device according to claim 28, wherein the integrated display, user interface and input means are in a touchscreen.

30. The device according to any one of claims 4-29, wherein the display, user interface and input means are discrete.

31. The device according to any one of claims 4-30, wherein the electronic program instructions comprise software.

32. The device according to claim 31, wherein the software is installed on a smartphone or provided as a software application downloadable to the smartphone.

33. The device according to any one of claims 4-32, wherein a computer-readable storage medium on which is stored instructions that, when executed by a computing means, causes the computing means to perform the method for calculating risk of a patient developing a serious infection.

34. A device according to any one of claims 4-33, wherein a computing means programmed to carry out the method for calculating risk of a patient developing a serious infection.

35. A device according to any one of claims 4-34, wherein the device comprises a data signal including at least one instruction being capable of being received and interpreted by a computing system, wherein the instruction implements the method for calculating risk of a patient developing a serious infection.

36. A system for calculating risk of a patient developing a serious infection comprising the device according to any one of claims 4-35 for calculating risk of a patient developing a serious infection.

37. A blood test to determine the risk of a patient suffering from rheumatoid arthritis developing a serious infection, wherein the blood test comprises measuring the concentration of MBL in the serum of a rheumatoid arthritis patient.
